(19) United States
(12) Patent Application Publication
(10) Pub. No.: US 2015/0272877 A1
(43) Pub. Date: Oct. 1, 2015

(54) KETOROLAC-CONTAINING SUSTAINED RELEASE DRUG DELIVERY SYSTEMS

(71) Applicant: ALLERGAN, INC., Irvine,, CA (US)

(72) Inventors: Ruiwen Shi, Irvine, CA (US); Patrick M. Hughes, Aliso Viejo, CA (US); Rhett M. Schiffman, Laguna Beach, CA (US)

(21) Appl. No.: 14/438,168

(22) PCT Filed: Oct. 24, 2013
(86) PCT No.: PCT/US2013/066644

§ 371 Date: Apr. 23, 2015

Related U.S. Application Data
(60) Provisional application No. 61/718,797, filed on Oct. 26, 2012.

(51) Int. Cl. A61K 9/00; A61K 47/34; A61K 47/10; A61K 31/407; A61K 47/26

(52) U.S. Cl. CPC A61K 9/0051; A61K 31/407; A61K 47/26; A61K 47/10; A61K 47/34

(57) ABSTRACT

Biodegradable intraocular and intraarticular drug delivery systems comprising ketorolac and a biodegradable polymer matrix that can release ketorolac into an eye or joint for an extended period of time are described. The drug delivery systems may be in the form of an extruded implant and may be used to treat one or more medical and ocular conditions, such as post-operative pain and inflammation following an ocular surgery such as cataract surgery.

### CROSS REFERENCE TO RELATED APPLICATIONS

This application claims the benefit of U.S. Provisional Patent Application Ser. No. 61/718,797, filed on Oct. 26, 2012, the entire disclosure of which is incorporated herein by reference.

### BACKGROUND

The present invention relates to biodegradable drug delivery systems that provide for the sustained release of ketorolac into an ocular tissue or joint. The drug delivery systems can be inserted into an ocular or intraarticular region to reduce pain and/or inflammation associated with a medical condition or a surgery, such as cataract surgery or refractive eye surgery.

Ketorolac is a non-steroidal anti-inflammatory drug that is often prescribed for the treatment of post operative pain and inflammation. However, effective management of the pain and inflammation associated with a surgery or medical condition can require frequent administration of ketorolac. In cases involving ocular surgery, patients may need to administer ketorolac-containing eye drops up to 4 times daily for 2 weeks. Furthermore, as many as four different eye drops may be prescribed by the doctor after an ocular surgery. As a result, patients may not only have difficulty complying with the dosing regimen but may also become confused by the different dosages and administration frequencies.

Nevertheless, ocular inflammation, if not effectively treated, can lead to vision damage. In some instances, macular edema may result. Thus, a biodegradable drug delivery system that can deliver a therapeutically effective amount of ketorolac directly into an eye for an extended period after only a single administration would be of great value to many different patients.

### SUMMARY

The present invention provides for biodegradable drug delivery systems that provide for the controlled and sustained release of ketorolac to an ocular region of the eye, such as the anterior chamber, posterior chamber, vitreous body, or to an intraarticular region of the body, such as a knee, elbow, wrist, or ankle joint, for the treatment of pain and inflammation or other inflammation-mediated conditions.

In one embodiment the drug delivery system comprises or consists of a biodegradable polymer matrix and ketorolac free acid, a pharmaceutically acceptable salt of ketorolac free acid, or a prodrug of ketorolac free acid associated with the biodegradable polymer matrix.

For example, in one embodiment the drug delivery system comprises ketorolac tromethamine (formula shown below) associated with a biodegradable polymer matrix.

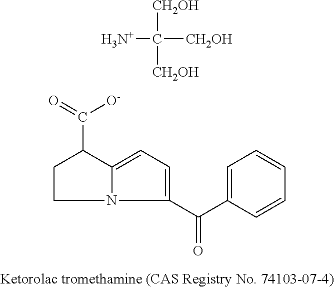 [SMILES: [NH3+]C(CO)(CO)CO ; O=C(c1ccccc1)c1ccc2n1CCC2C(=O)[O-]]

In another embodiment, the drug delivery system comprises ketorolac free acid associated with a biodegradable polymer matrix.

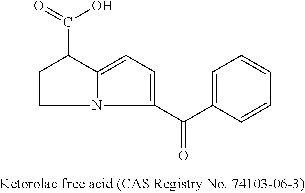 [SMILES: O=C(c1ccccc1)c1ccc2n1CCC2C(=O)O]

In another embodiment, the drug delivery system comprises an ester or amide prodrug of ketorolac free acid associated with a biodegradable polymer matrix.

In another embodiment the drug delivery system comprises ketorolac free acid, a prodrug of ketorolac, or a pharmaceutically acceptable salt of ketorolac as the pharmaceutically active agent and a biodegradable polymer matrix and comprises no pharmaceutically active agent other than ketorolac.

The drug delivery system can comprise or consist of a plurality of microspheres or a solid or semi-solid implant configured for intracameral (i.e. anterior chamber), posterior chamber (i.e. behind the iris in the ciliary sulcus), intravitreal, anterior vitreal, sub-retinal, suprachoroidal, intrascleral, subconjunctival, periocular, or sub-Tenon's space administration to a patient suffering from an ocular condition, including ocular pain and/or inflammation, to thereby treat the ocular condition. The ocular pain and/or inflammation may result from or be associated with cataract surgery. Examples of solid implants include extruded implants (sometimes referred to as fibers, filaments, or rods) and compressed tablets. An extruded implant can be cylindrical (such as a rod) or non-cylindrical. Thus, an extruded biodegradable implant is one example of a drug delivery system within the scope of the present invention.

An extruded biodegradable implant according to this invention can be configured for placement in the anterior chamber, posterior chamber, or vitreous body of an eye. Such an implant may be generally referred to as an intraocular implant. For example, an intraocular drug delivery system according to the present disclosure can be in the form of an extruded biodegradable intraocular implant configured for placement in the anterior chamber of an eye of a human or non-human mammal.

In another embodiment, the drug delivery system can comprise an extruded implant or plurality of microspheres for placement in a joint to treat pain and/or inflammation in a joint or a medical condition of the joint such as arthritis in a mammal in need thereof. Such an implant may be referred to as an intraarticular implant.

The intraocular or intraarticular implant can be biodegradable and can comprise or consist of a biodegradable polymer matrix and ketorolac free acid, a pharmaceutically acceptable salt of ketorolac free acid such as ketorolac tromethamine, or a prodrug of ketorolac associated with the biodegradable polymer matrix. The ketorolac can be homogenously or heterogeneously dispersed and/or dissolved in the biodegradable polymer matrix.

An intraocular or intraarticular implant according to this disclosure is loaded with sufficient ketorolac to release a therapeutically effective amount of ketorolac into the eye (or joint) of the patient over an extended period of time, which may be from about 1 day to about 6 weeks, for about 2 weeks or more, 3 weeks or more, for about one month or more, about 6 weeks or more, or for about 1 week to about 52 weeks. Yet, the implant is sized and configured to be comfortable and non-irritating to the eye of the patient.

An implant sized and configured for administration to the anterior chamber can be about 0.5 mm to about 3 mm in length, about 100 μm to about 750 μm in diameter, and about 50 μg to about 1000 μg, or more specifically about 50 μg to about 300 μg in total weight. In one embodiment, the diameter (or other smallest dimension as in the case of non-cylindrical filaments) of an anterior chamber implant is about 100 μm to about 500 μm. In one embodiment the total weight of the anterior chamber implant is from about 100 μg to about 500 μg. For example, the anterior chamber implant can weigh about 100 μg, about 150 μg, about 200 μg, about 250 μg, about 300 μg, about 400 μg, or about 500 μg. Posterior chamber implants may have the same dimensions and weights as an anterior chamber (intracameral) implant.

These implants can be used, for example, to reduce ocular pain and inflammation resulting from ocular surgery such as cataract surgery or refractive eye surgery. These implants promote healing of inflamed tissue and may further lower the risk of post-surgical complications, including macular edema. Thus, implants may be placed in the anterior or posterior chambers of an eye, or vitreous body during an ocular surgery such as during cataract surgery to treat inflammation and pain following the surgery or may be placed in the eye without surgery to treat an anterior or posterior ocular condition of the eye in a mammal

The release of ketorolac from the intraocular implant comprising a biodegradable polymer matrix can include an initial burst of release of ketorolac followed by a gradual increase in the amount of ketorolac released, or may include an initial delay in release of the ketorolac followed by an increase in release, or, in some cases, the implant may provide a steady, constant rate of release of ketorolac for an extended period of time, which can be 3 weeks or more. An initial drug burst followed by a lower maintenance drug release may be especially beneficial for treating a number of ocular inflammatory conditions, particularly post-operative pain and inflammation, as may occur after cataract surgery.

Accordingly, the present invention provides for implants that release a therapeutically effective amount of ketorolac into the eye of a patient (which can be a human or non-human mammal) in a pre-defined manner over an extended period of time, for example, over a period of time between about 1 day and about 6 weeks. The pre-defined manner of ketorolac release from the implant may consist of an initial rapid release phase (usually lasting about 2 to 24 hours) followed by a slower release phase (lasting for about 3 weeks or more). In this regard, the present invention provides for an extruded intraocular or intraarticular implant that releases about 5 μg to about 200 μg of ketorolac within 24 hours (day 1) after placement in an ocular region of the eye and from about 0 μg to about 5 μg, from about 0.001 μg to about 5 μg, from about 0.01 μg to about 5 μg, from about 0.05 μg to about 5 μg, or from about 0.1 μg to about 5 μg of ketorolac per day each day thereafter (that is after day 1 or after about t=24 hours, where t is time) for about 3 weeks (21 days) or more after placement in the eye. Ketorolac released in this manner can be an amount of ketorolac effective for reducing pain and inflammation in an eye for an extended period (e.g, at least about three weeks or more) following cataract surgery.

In some embodiments the extruded implant releases about 5-100 μg, 10-50 μg, 20-100 μg, 30-100 μg, 20-70 μg, 30-60 μg, or about 50-100 μg of ketorolac within 24 hrs (day 1) after placement in an ocular region of the eye and from about 0.1 μg to about 5 μg, about 0.5-3 μg, about 1 to about 2 μg, or about 0.2 to about 2 μg of ketorolac per day each day thereafter for about 2 or 3 weeks or more. In some formulations, the post-24 hour release continues at any of these rates for 28 days or more, such as, for example, about 6 weeks.

In accordance with any of the foregoing embodiments the implant can be formulated such that it releases at least 1%, at least 5%, or at least 10% of its initial ketorolac load but no more than 50% of its initial ketorolac load during the first 60 minutes following placement of the implant in an ocular region of an eye of a mammal.

In some cases, such as in the treatment of an infection or chronic condition such as macular edema, it may be desirable to provide a relatively constant rate of release of ketorolac from the implant over the life of the system. For example, it may be desirable for the ketorolac to be released in amounts from about 0.01 μg to about 2 μg or more per day for the life of the system.

In one embodiment a biodegradable intraocular implant effective for reducing pain and/or inflammation in an eye of a mammal can comprise from about 10% to about 60% by weight ketorolac and about 40% to about 90% by weight poly(lactide-co-glycolide), polylactide, or a combination thereof, wherein the diameter or smallest dimension of the implant is about 0.1 mm to about 1.5 mm, and wherein the implant releases about 1 μg to about 400 μg of ketorolac within the first 24 hours of its insertion into an ocular region of a mammal, and about 0.001 μg to about 5 μg/day thereafter for about 2 weeks, 3 weeks, or 2 to 6 weeks after placement of the implant in the ocular region of the mammal. The intraocular implant may comprise about 40% to about 85% by weight of poly (lactide-co-glycolide), polylactide, or a combination thereof, may be rod-shaped, and may have a diameter (or other smallest dimension) of about 0.2 mm to about 1.0 mm and a length of about 0.5 mm to about 3 mm. An implant according to this embodiment may release about 5 μg to about 200 μg of ketorolac during the first 24 hours and from about 0, 0.01, 0.05, 0.1, or 0.5 μg to about 5 μg/day thereafter for about 2, 3, or 6 weeks or more after placement of the system in the eye of the mammal.

In addition to a biodegradable polymer matrix and ketorolac, an implant may optionally further comprise about 0.1% to about 10% by weight of a polyethylene glycol or polyethylene oxide with a molecular weight of about 300 to about 40,000; or about 1% to about 10% by weight of polyethylene glycol with a molecular weight of about 3350; or about 1% to about 10% by weight of polyethylene glycol or polyethylene oxide with a molecular weight of about 20,000; and/or about 0.1% to about 10% by weight of a low molecular weight water soluble substance such as trehalose, sucrose, dextrose, or mannitol.

Any of the drug delivery systems described herein advantageously provide for extended release times of ketorolac. Thus, the patient, in whose eye (or joint) the drug delivery system has been placed, receives a therapeutic amount of ketorolac for an extended time without requiring additional administrations of ketorolac as is typically required with topical formulations. For example, extruded implants or microspheres of the present invention, upon placement in the anterior chamber of an eye, can deliver a therapeutically effective amount of ketorolac to the iris-ciliary body in the eye for at least about one day, at least about one week, two weeks, between about one week and six months, between about one day and about six weeks, or for at least about 6 weeks after receiving an implant or microparticles. The sustained local delivery of the therapeutic agent from the present drug delivery systems reduces the high transient concentrations associated with traditional bolus injection or pulsed dosing. Furthermore, direct intracameral or intravitreal administration of the present systems obviates the constraints posed by the blood-retinal barrier and significantly reduces the risk of systemic toxicity.

Ocular conditions that can be treated by ketorolac-containing drug delivery systems according to the present invention include ocular pain and inflammation, such as post-operative ocular pain and inflammation resulting from an ocular surgery such as cataract surgery or refractive eye surgery; macular edema, diabetic macular edema, chronic diabetic macular edema, uveitis (anterior, intermediate, or posterior), exudative or non-exudative age-related macular degeneration (AMD), diabetic retinopathy, proliferative vitreal retinopathy, retinal vein occlusion, central retinal vein occlusion (CRVO), and branch retinal vein occlusion (BRVO).

Some embodiments provide for a method of treating ocular pain and/or inflammation comprising placing a drug delivery system, such as an extruded biodegradable intraocular implant, into an ocular region of an eye in a mammal in need thereof. In some forms of this method the drug delivery system is placed in the anterior chamber, posterior chamber, or vitreous body of the eye of the mammal.

Some embodiments provide for a method of treating pain and inflammation in an eye after cataract surgery, comprising placing a drug delivery system described herein (such as an extruded biodegradable implant) into an ocular region of the eye of a patient during cataract surgery, thereby reducing pain and inflammation associated with the surgery. In some forms of this method, the drug delivery system is placed in the anterior chamber or posterior chamber of the eye during cataract surgery. The method may have the added benefit of lowering the risk of the mammal developing post-operative macular edema. The method can include making an incision in the eye and placing a ketorolac-containing implant of the present invention into an ocular region of the eye opened by the incision, wherein the incision is made in the eye as part of a cataract surgery. The implant may not only reduce post-operative pain and/or inflammation arising from cataract surgery but also provide for shorter recovery times and more rapid improvement in vision as compared to the administration of ketorolac solely by use of eye drops. The method can comprise placing the ketorolac-containing implant into the anterior chamber of the eye during cataract surgery. Alternatively, or in addition, the method can comprise placing a ketorolac-containing implant into the posterior chamber or vitreous body of the eye during cataract surgery.

Some embodiments provide for a method of treating macular edema in an eye of a mammal in need thereof, comprising placing a drug delivery system described herein into an ocular region of the eye of the mammal, thereby treating the macular edema and, possibly, improving the visual performance of the eye. The macular edema treated by this method can be a chronic diabetic macular edema.

Accordingly, the drug delivery systems described herein (which can comprise or consist of an extruded ketorolac-containing biodegradable implant or plurality of microspheres) may be useful not only for reducing pain and inflammation in an eye following cataract surgery on the eye, but also for reducing the risk of developing macular edema after cataract surgery.

In addition, the drug delivery systems described herein may also be useful for treating inflammation in a joint, comprising injecting, implanting, or otherwise administering a ketorolac-containing implant or plurality of microspheres comprising ketorolac, as described herein, into an inflamed joint or area adjacent to the inflamed joint. The inflammation in the joint may be due to a surgery on the joint or a medical condition of the joint such as arthritis.

The invention further provides for a method of making a ketorolac-containing drug delivery system, comprising combining or mixing ketorolac with a biodegradable polymer or two or more biodegradable polymers. The mixture may then be extruded or compressed to form a single composition. The single composition may then be processed (e.g., cut to a desired length or size and sterilized) to form individual implants suitable for placement in an eye of a patient. Another method may involve an emulsion/solvent evaporation process, including but not limited to an oil in water emulsion process, which may be useful in producing ketorolac-containing microspheres.

### BRIEF DESCRIPTION OF THE DRAWINGS

FIG. 1 illustrates diagrammatically a cross-sectional view of an eye.

FIG. 2 shows the cumulative release of ketorolac over time for Implants 6-1 to 6-6.

FIG. 3 shows the cumulative release of ketorolac over time for Implants 6-7 to 6-9.

FIG. 4 shows the cumulative release of ketorolac over 24 hours for Implants 6-6 to 6-8.

FIG. 5 shows the cumulative release of ketorolac over 24 hours for Implants 7-3 to 7-5.

FIG. 6 shows the in vitro cumulative release data for implants 6-6, 6-8, 6-9, 7-3, 7-4, and 7-5 from Examples 3 and 4.

### DETAILED DESCRIPTION

“Active agent”, “drug”, “therapeutically active agent,” and “pharmaceutically active agent” refer to the chemical compound that produces a therapeutic effect in the patient (human or non-human mammal) to which it is administered and that can be used to treat a medical condition, such as an ocular condition or an adverse condition of a joint in the body, such as pain or inflammation of a joint. One example of a therapeutically active agent in the context of the present invention is ketorolac.

A “patient” can be a human or non-human mammal.

A drug delivery system according to the present disclosure may comprise a prodrug of ketorolac. A “prodrug” means a compound (e.g., a drug precursor) that is transformed in vivo to yield an active form of the compound. The transformation may occur by various mechanisms (e.g., by metabolic or chemical processes), such as, for example, through hydrolysis. Examples of useful ketorolac prodrugs include esters of ketorolac comprising a linear or branched alkyl group such as, for example, a methyl, ethyl, propyl, isopropyl, butyl, isobutyl, or other (C₁-C₈)alkyl. Examples are disclosed in Doh et al. (2003) “Synthesis and Evaluation of Ketorolac Ester Prodrugs for Transdermal Delivery” J. Pharmaceutical Sciences, Vol. 92, No. 5.

Additional examples of useful ketorolac prodrugs include amides formed by replacement of the —OH group of the carboxylic acid group in ketorolac with an amine or group of formula —NR^{x}R^{y}, wherein R^{x} and R^{y} can be the same or independently H, alkyl, aryl, cycloalkyl, cycloalkenyl, or heterocycle. Examples are disclosed in Kim et al. (2005) “Ketorolac amide prodrugs for transdermal delivery: stability and in vitro rat skin permeation studies” International Journal of Pharmaceutics Volume 293, Issues 1-2, Pages 193-202.

The term “alkyl”, as used herein, refers to saturated monovalent or divalent hydrocarbon moieties having linear or branched moieties or combinations thereof and containing 1 to 6 carbon atoms. One methylene (—CH₂—) group, of the alkyl can be optionally replaced by oxygen, sulfur, sulfoxide, nitrogen, carbonyl, carboxyl, sulfonyl, amide, sulfonamide, by a divalent C₃₋₆ cycloalkyl, by a divalent heterocycle, or by a divalent aryl group. Non-limiting examples of suitable alkyl groups include methyl (—CH₃), ethyl (—CH₂CH₃), n-propyl (—CH₂CH₂CH₃), isopropyl (—CH(CH₃)₂), and t-butyl (—C(CH₃)₃).

A “joint” as used herein refers to the point of contact between two or more bones of an animal or human skeleton with the parts that surround and support it. Examples of joints include without limitation the knee joint, toe and finger joints, wrist, ankle, hip, shoulder, back (vertebrae and vertebral discs), and elbow.

An “intraarticular region” refers to a joint, such as a knee, elbow, shoulder, finger, toe, or hip joint. Intraarticular regions include joints in the wrist and vertebral column in the neck and back.

“Associated with a biodegradable polymer matrix” means mixed with, dissolved and/or dispersed within, encapsulated by, surrounded and/or covered by, or coupled to.

The term “biodegradable polymer” refers to a polymer or polymers which degrade in vivo, and wherein degradation of the polymer or polymers over time occurs concurrent with or subsequent to release of the therapeutic agent. A biodegradable polymer may be a homopolymer, a copolymer, or a polymer comprising more than two different structural repeating units.

An “intraocular implant” refers to a device or element that is configured to be placed in an ocular region of the eye. Examples include extruded filaments, comprising a biodegradable polymer matrix and an active agent, such as ketorolac, associated with the polymer matrix, and cut to a length suitable for placement in an eye. Intraocular implants are generally biocompatible with the physiological conditions of an eye and do not cause adverse reactions in the eye. In certain forms of the present invention, an intraocular implant may be configured for placement in the anterior chamber, posterior chamber, or vitreous body of the eye. Intraocular implants may be placed in an eye without significantly disrupting vision of the eye. Implants can be biodegradable and may be produced by an extrusion process, as described herein. Implants produced by an extrusion process and comprising a biodegradable polymer matrix and ketorolac free acid or a prodrug or pharmaceutically acceptable salt thereof, such as ketorolac tromethamine, are examples of a drug delivery system within the scope of the present invention.

An “intracameral” implant is an implant that is sized, configured, and formulated for placement in the anterior chamber of the eye.

An “intravitreal” implant is one that is sized, configured, and formulated for placement in the vitreous body of the eye.

The term “biocompatible” means compatible with living tissue or a living system. Biocompatible implants and polymers produce few or no toxic effects, are not injurious, or physiologically reactive and do not cause an immunological reaction.

“Cumulative release profile” means the cumulative total percent of an active agent (such as ketorolac) released from an implant into an ocular region or site in vivo over time or into a specific release medium in vitro over time.

“Suitable (or configured) for insertion, implantation, or placement in (or into) an ocular region or site” with regard to an implant, means an implant which has a size (dimensions) such that it can be inserted, implanted, or placed in an eye without causing excessive tissue damage or physically impairing the existing vision of the patient into which the implant is implanted or inserted.

“Treating” and “treatment” as used herein includes any beneficial effect in the eye or intraarticular region of an individual produced by the present methods. Treatment of an ocular or intraarticular condition may reduce, or retard the progression of, one or more signs or symptoms of the ocular or intraarticular condition. The sign(s) or symptom(s) positively affected by the treatment will depend on the particular condition. Examples of beneficial (and therefore positive) effects produced by the present methods may include but are not limited to a reduction in pain, burning and/or foreign body sensation, itching, redness, swelling, inflammation, and/or discomfort.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of an ocular region in an eye include the anterior chamber, the posterior chamber, the vitreous cavity (sometimes referred to as the vitreous body or the vitreous), the choroid, the suprachoroidal space, the conjunctiva, the subconjunctival space, the sub-Tenon's space, the episcleral space, the intracorneal space, the epicorneal space, the sclera, the pars plana, surgically-induced avascular regions, the macula, and the retina.

The anterior chamber refers to the space inside the eye between the iris and the innermost corneal surface (endothelium).

The posterior chamber refers to the space inside the eye between the back of the iris and the front face of the vitreous (that is, the small space directly posterior to the iris but anterior to the lens). The posterior chamber includes the space between the lens and the ciliary process, which produces the aqueous humor that nourishes the cornea, iris, and lens and maintains intraocular pressure.

The term “pharmaceutically acceptable salts” refers to salts or complexes that retain the desired biological activity of the compound (such as ketorolac) and exhibit minimal or no undesired toxicological effects to the mammal or cell system to which they are administered. The “pharmaceutically acceptable salts” according to the invention include therapeutically active salt forms of ketorolac. Useful pharmaceutically acceptable salts can include those formed by treating ketorolac free acid with sodium hydroxide, magnesium hydroxide, potassium hydroxide, calcium hydroxide, ammonia and the like; or an organic base such as for example, L-arginine, ethanolamine, betaine, benzathine, morpholine, tromethamine, and the like. Salts formed with zinc are also of potential interest.

As used herein, an “ocular condition” is a disease, ailment, or condition which affects or involves the eye or one of the parts or regions of the eye, including the anterior or posterior regions of the eye. The eye is the sense organ for sight. Broadly speaking the eye includes the eyeball and the tissues and fluids which constitute the eyeball, the periocular muscles (such as the oblique and rectus muscles) and the portion of the optic nerve which is within or adjacent to the eyeball.

Non-limiting examples of an ocular condition include ocular pain and/or inflammation resulting from, for example, ocular surgery (therefore, post-operative ocular pain and inflammation). Drug delivery systems according to the present disclosure may be used to reduce and, potentially, prevent pain and/or inflammation resulting from and associated with cataract surgery. Cataract surgery may cause inflammation of certain ocular tissues, including the iris and ciliary body. This inflammation may give rise to ocular pain. The inflammation and pain associated with cataract surgery may be effectively reduced, and thereby treated, by administration of a ketorolac-containing drug delivery system described herein.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the ciliary body, the posterior chamber, the lens or the lens capsule and blood vessels and nerve which vascularize or innervate an anterior ocular region or site.

Thus, an anterior ocular condition can include pain and/or inflammation of the eye resulting from cataract surgery, or a disease, ailment or condition, such as for example, aphakia; pseudophakia; astigmatism; blepharospasm; cataract; conjunctival diseases; conjunctivitis; corneal diseases; corneal ulcer; dry eye syndrome; eyelid diseases; lacrimal apparatus diseases; lacrimal duct obstruction; myopia; presbyopia; pupil disorders; refractive disorders; pain and inflammation; an inflammatory condition; and strabismus. One example of an inflammatory condition is inflammation of the ciliary body. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as the choroid or sclera (in a position posterior to a plane through the posterior wall of the lens capsule), vitreous body, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration; exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, or radiation retinopathy; epiretinal membrane disorders; branch retinal vein occlusion; anterior ischemic optic neuropathy; non-retinopathy diabetic retinal dysfunction; retinitis pigmentosa; and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

An “intraarticular condition” is a disease, ailment, or adverse condition that affects or involves an intraarticular region of the body and that may impair the normal function or use of that region of the body. Non-limiting examples of intraarticular conditions include arthritis, pain, and inflammation.

“Inflammation-mediated” in relation to an ocular condition means any condition of the eye which can benefit from treatment with an anti-inflammatory agent such as ketorolac and is meant to include, but is not limited to uveitis, macular edema, acute macular degeneration, retinal detachment, ocular tumors, fungal, bacterial, or viral infections, multifocal choroiditis, diabetic uveitis, proliferative vitreoretinopathy (PVR), sympathetic opthalmia, Vogt Koyanagi-Harada (VKH) syndrome, histoplasmosis, uveal diffusion, and inflammation in the eye due to cataract surgery.

The term “therapeutically effective amount” or “effective amount” refers to the level or amount of active agent needed to treat an ocular or intraarticular condition without causing significant negative or adverse side effects to the eye or a region of the eye or body to which the agent is administered.

### DESCRIPTION

Controlled and sustained administration of ketorolac through the use of one or more of the intraocular or intraarticular drug delivery systems described herein, such as one or more ketorolac-containing implants or microspheres, may improve treatment of an undesirable ocular or intraarticular condition, and can reduce pain and/or inflammation resulting from an ocular surgery or intraarticular surgery (surgery on a joint such as arthroscopic surgery).

The drug delivery systems can comprise a biodegradable polymer matrix and are formulated to release ketorolac over an extended period of time. The intraocular and intraarticular drug delivery systems are effective to provide a therapeutically effective amount of ketorolac directly to an ocular region of the eye or into an intraarticular region of the body, such as a joint, to treat, prevent, and/or reduce one or more undesirable ocular or medical conditions. Thus, with a single administration of the drug delivery system, ketorolac will be made available at the site where it is needed and will be maintained for an extended period of time, rather than subjecting the patient to repeated injections or, in the case of self-administered eye drops, the burden of dosing multiple times every day and ineffective treatment with only limited bursts of exposure to the active agent, or in the case of systemic administration, higher systemic exposure and concomitant side effects or, in the case of non-sustained release dosages, potentially toxic transient high tissue concentrations associated with pulsed, non-sustained release dosing.

An improvement of the ocular or intraarticular condition obtained by use of drug delivery system described herein may be observed or perceived by a reduction in pain, redness, and or swelling and/or by a general feeling of comfort. The improvement in the ocular conditions may further be observed by an improved visual performance by the patient.

An intraocular drug delivery system in accordance with the disclosure herein comprises ketorolac and a biodegradable polymer matrix. The drug delivery system may be monolithic, i.e. having the active agent (for example ketorolac) or agents homogenously distributed through the polymeric matrix. Alternatively, the active agent may be distributed in a non-homogenous pattern in the polymer matrix. For example, an implant may include a portion that has a greater concentration of the ketorolac component relative to a second portion of the implant. One example of a drug delivery system within the scope of the present invention is an extruded biodegradable implant comprising or consisting of ketorolac and a biodegradable polymer matrix. The implant may sustain release of a therapeutically effective amount of the ketorolac in a pre-defined manner into an eye in which the implant is placed. As previously discussed, the pre-defined manner of ketorolac release from the implant may consist of a fast release phase followed by a slower release phase. The fast release phase refers to the rate of ketorolac release during the first 24 hours after placement of the implant in the eye (that is, from t=0 hr to about t=24 hr, where t is time). The rate of ketorolac release can be expressed as the mass of ketorolac released over a period of time. In the fast release phase, the implant releases ketorolac at a substantially high and consistent rate over a period of about 24 hours. In the slower release phase the implant releases ketorolac at a rate substantially lower than that observed during the first 24 hrs (the fast release phase). The slower release phase refers to the period beginning on about the second day (therefore, on day 2, beginning at about t=24 hrs) after placement of the implant in a patient, and lasting for about 1 day to about 6 weeks or more thereafter.

The release of ketorolac according to the pre-defined manner described above, whereby the patient receives an initial burst or higher dose of the drug followed by a lower maintenance dose of the drug for an extended period (i.e, fast release phase followed by slower release phase), may be especially effective for treating the pain and inflammation associated with cataract surgery. Accordingly, the present invention describes drug delivery systems (such as extruded implants) that will release ketorolac in this manner upon placement in the anterior chamber of the eye to reduce the pain and inflammation resulting from cataract surgery. Examples of such drug delivery systems may include the extruded biodegradable implants corresponding to Implant Nos. 6-1, 6-3, 6-6, 6-9, and 7-3, described in Tables 1, 2, and 4, below.

In some embodiments, the rate of release of ketorolac from the implant during the fast release phase is at least about 2× (two times), about 5×, about 10×, about 20×, about 50×, about 100×, about 1000×, or about 10,000× greater than the rate of release of ketorolac during the slower release phase. The release rate may be expressed as, for example, mass of ketorolac released during a specified time period. For example, the implant may release about 5 μg to about 200 μg of ketorolac during the first 24 hrs (therefore, on day 1) after placement in an ocular region of an eye and from about 0 μg to about 5 μg of ketorolac per day each day thereafter (that is, after day 1) for about 1 day to about 2, 3, or 6 weeks or more. In some embodiments, the implant releases about 0.001 μg to about 5 μg, from about 0.01 μg to about 5 μg, from about 0.05 μg to about 5 μg, or from about 0.1 μg to about 5 μg of ketorolac per day after day 1 for about 3 weeks (21 days) or more. In one embodiment an implant according to the invention releases about 10 μg to about 100 μg of ketorolac during the first 24 hours in the eye (i.e, during the fast release phase) and about 0.5 to about 3 μg of ketorolac/day each day thereafter (i.e, during the slower release phase) for about 14 days (two weeks) or more, 21 days or more, about 28 days or more, about 35 days or more, or about 42 days. The ocular region can be the anterior chamber.

Biodegradable Implants for Treating an Ocular Condition

Biodegradable implants according to the present invention may comprise or consist of i) a biodegradable polymer matrix and ii) ketorolac free acid, a pharmaceutically acceptable salt of ketorolac free acid, such as ketorolac tromethamine, or a prodrug of ketorolac free acid associated with the biodegradable polymer matrix. The biodegradable implant can be produced by an extrusion process and may therefore be an extruded cylindrical or non-cylindrical filament in which the ketorolac is associated with biodegradable polymer matrix. The extruded filament can have a diameter and be cut to a length suitable for placement in an intraocular or intraarticular region.

The implant may comprise about 10% to about 60%, about 20% to about 60%, or about 30% to about 50% ketorolac (free acid, salt, or prodrug) by weight with the remaining weight being made up by one or more biodegradable polymers, and optionally one or more excipients. In some embodiments, the implant may comprise about 20 μg to about 250 μg, or about 30 μg to about 150 μg ketorolac free acid, ketorolac salt (for example, ketorolac tromethamine), or ketorolac prodrug.

The biodegradable polymer matrix may comprise one, two, or more poly(D,L-lactide-co-glycolide) copolymers and/or one, two, or more poly(D,L-lactide) polymers, including but not limited to any of the particular RESOMER® PLGA and PLA polymers disclosed herein. The implant may be effective in sustaining release of an amount of ketorolac therapeutically effective for the treatment of an ocular condition, such as pain and inflammation in an eye, for a time period from about one day to about three or six weeks, or for about one month to about four months, from the time the system is placed in an eye.

In addition to ketorolac and one or more biodegradable PLGA and/or PLA polymers, an implant may optionally further comprise one or more excipients to improve the properties of the implant. Useful excipients include preservatives, anti-oxidants, buffering agents, chelating agents, electrolytes (e.g. NaCl, KCl, or MgCl₂), polyethylene glycols, and low molecular weight water soluble substances. Different excipients may be combined. For example, an implant may optionally further comprise about 0% to about 10% by weight of a low molecular weight water soluble substance such as a saccharide. Useful saccharides include trehalose, sucrose, dextrose, and mannitol. In some embodiments, an implant may optionally further comprise about 1% to about 20% by weight of a polyethylene glycol (PEG) or polyethylene oxide (PEO). An implant may comprise both a polyethylene glycol and a saccharide. Useful polyethylene glycols include PEG 3350 and PEG 20,000.

For implantation in an ocular region, the total weight of the implant may vary, from, for example, about 20 μg to about 15000 μg, about 100 μg to about 5000 μg, about 120 μg to about 1,800 μg, about 2400 μg to about 3,600 μg, about 100 μg to about 2 mg, or about 50 μg to 1 mg. The total weight of an anterior (intracameral) or posterior chamber implant may be about 20 μg to about 400 μg, about 30 μg to about 300 μg, about 50 μg to about 250 μg, about 50 to about 300 μg, about 100 μg to about 400 μg, or about 50, 100, 120, 150, 190, 200, 250, or about 300 μg.

The upper limit for the implant size will be determined by factors such as the desired release kinetics, toleration for the implant at the site of implantation, size limitations on insertion, and ease of handling. For example, the vitreous body and other parts of an eye are able to accommodate relatively large rod-shaped implants, generally having diameters of about 0.5 mm to 3 mm and a length of about 5 to about 10 mm, while the anterior and posterior chambers of the eye will require smaller implants.

Implants configured for insertion into the anterior chamber (intracameral implants) may have a diameter or other smallest dimension (as may be appropriate for non-cylindrical implants) of about 100 μm to about 1 mm and a length of about 0.5 mm to about 3 mm. Some intracameral implants may have a diameter or other smallest dimension (as appropriate for non-cylindrical implants) of about 50 μm to about 500 μm, about 100 μm to about 500 μm, about 50 μm to about 300 μm, or about 50 μm to about 200 μm. In some embodiments, an intracameral implant may have a diameter or other smallest dimension (as in the case of non-cylindrical implants) of about 200 μm (corresponding to a 28 gauge needle) to about 360 μm (corresponding to a 25 gauge needle). Intracameral implants may have a length of about 0.5 mm to about 3 mm. In a particular embodiment the intracameral implant has a diameter of about 50 μm to about 500 μm (i.e., about 50-500 μm) and a length of about 0.5 mm to about 2.5 mm. In some embodiments an intracameral implant may have a diameter of about 50 μm to about 500 μm and a length of about 0.5 mm to about 3 mm.

In some embodiments, an implant configured for administration to the anterior chamber (an intracameral implant) is about 0.5 mm to about 2 mm (i.e., about 0.5-2 mm) in length, about 100 μm to about 750 μm in diameter, and about 50 μg to about 1000 μg, or more specifically about 50 μg to about 300 μg in total weight. In specific forms the diameter (or other smallest dimension as in the case of non-cylindrical filaments) and total weight of the intracameral implant is about 100 μm to about 500 μm and about 100 μg to about 500 μg, respectively. For example, the intracameral implant can weigh about 100 μg, about 150 μg, about 190 μg, about 200 μg, about 250 μg, or about 300 μg, or from about 120 μg to about 400 μg. In some embodiments, an intracameral implant may have a length of from about 0.5 mm to about 2.5 or 3.5 mm, a diameter of from about 100 μm to about 500 μm, and a total weight of from about 100 μg to about 400 μg.

Biodegradable Polymers

In one aspect, the present invention provides for extruded biodegradable intraocular and intraarticular implants comprising a biodegradable polymer matrix and ketorolac free acid, ketorolac tromethamine, or a ketorolac prodrug associated with the biodegradable polymer matrix. The intraocular and intraarticular implants according to the present invention are generally biocompatible with the physiological conditions of an eye or joint and do not cause unacceptable adverse side effects in the eye or joint. Thus, the biodegradable polymer matrix will generally comprise one or more biodegradable polymers that are biocompatible with the eye or joint so as to cause no substantial interference with the functioning or physiology of the eye or joint. Such polymers are preferably at least partially and more preferably substantially completely biodegradable or bioerodible. Bioerodible polymers include those that dissolve in vivo.

Useful biodegradable polymers include poly(D,L-lactide) (PLA) polymers and poly(D,L-lactide-co-glycolide) (PLGA) copolymers. In general, the biodegradable polymer matrix may comprise a PLA polymer, a PLGA copolymer, a mixture of two or more different PLA polymers, a mixture of two or more different PLGA copolymers, or a combination of one, two, or more PLA polymers and one, two, or more PLGA copolymers. For example, the biodegradable polymer matrix may comprise one or more poly(D,L-lactide-co-glycolide) copolymers and/or one or more poly(D,L-lactide) polymers. For example, the polymer matrix may comprise or consist of one poly(D,L-lactide) polymer and/or one poly(D,L-lactide-co-glycolide) copolymer, or the implant may comprise two or more different poly(D,L-lactide) polymers and/or one, or two or more different poly(D,L-lactide-co-glycolide) copolymers. A polymer or copolymer may differ from another polymer or copolymer with regard to the end group, inherent viscosity, or repeating unit of the polymer, or any combination of thereof. For example, when two poly(D,L-lactide) polymers are present, the first PLA polymer may have a first inherent viscosity and an acid end group while the second PLA polymer may have a second inherent viscosity (different from the first) and an ester end group. Thus, in addition to one, two, or more poly(D,L-lactide) polymers, an implant may comprise one, two, or more poly(D,L-lactide-co-glycolide) copolymers. When two poly(D,L-lactide-co-glycolide) copolymers are present, the first poly(D,L-lactide-co-glycolide) copolymer may have a first inherent viscosity and an ester end group and the second poly(D,L-lactide-co-glycolide) copolymer may have a second inherent viscosity (different from the first) and an acid end group. Alternatively, the first and second poly(D,L-lactide-co-glycolide) copolymers may each have an ester end group.

Polylactide, or PLA, includes poly(L-lactide), poly(D-lactide), and poly(D,L-lactide). Poly (D,L-lactide) may also be identified by CAS Number 26680-10-4, and may be represented by the formula:

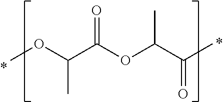

Poly(lactide-co-glycolide), or PLGA, includes poly(D,L-lactide-co-glycolide), also identified by CAS Number 26780-50-7, and may be represented by a formula:

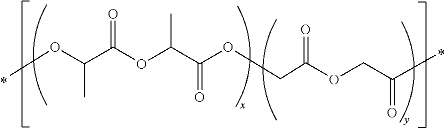

Thus, poly(D,L-lactide-co-glycolide) comprises one or more blocks of D,L-lactide repeat units (x) and one or more blocks of glycolide repeat units (y), where the size and number of the respective blocks may vary. The molar percent of each repeat unit in a poly(lactide-co-glycolide) (PLGA) copolymer may be independently 0-100%, about 15-85%, about 25-75%, or about 35-65%. In certain variations, 25/75 PLGA and/or 50/50 PLGA copolymers are used. In some embodiments, the D,L-lactide may be about 50% to about 75% of the PLGA polymer on a molar basis, such as: about 48% to about 52%, or about 50%; or about 73% to about 77%, or about 75%. The balance of the polymer may essentially be the glycolide repeat units. For example, the glycolide may be about 25% to about 50% of the PLGA polymer on a molar basis, such as: about 23% to about 27%, or about 25%; or about 48% to about 52%, or about 50%.

Biodegradable polymer matrices that include mixtures of PLA and PLGA polymers may be employed, and are useful in modulating the release rate of ketorolac free acid or ketorolac tromethamine. The PLA and PLGA polymers may be hydrophobic ended (also referred to as capped or end-capped), having an ester linkage hydrophobic in nature at the polymer terminus, or hydrophilic ended (also referred to as uncapped), having an end group hydrophilic in nature at the polymer terminus, such as a carboxylic acid end group. Typical hydrophobic end groups include, but are not limited to alkyl esters and aromatic esters. Hydrophilic end groups at the polymer terminus degrade faster than hydrophobic ended PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. A specific end group may result from the initiator employed in the polymerization process. For example, if the initiator is water or carboxylic acid, the resulting end groups may be carboxyl and hydroxyl. Similarly, if the initiator is a monofunctional alcohol, the resulting end groups may be ester or hydroxyl.

The selection of the biodegradable polymer(s) used to prepare an implant can vary with the desired release kinetics, patient tolerance, the nature of the disease to be treated, and the like. Polymer characteristics that are considered include, but are not limited to, the biocompatibility and biodegradability at the site of implantation, molecular weight and molecular weight distribution, hydrophilicity or hydrophobicity, compatibility with the active agent, and processability. The biodegradable polymer matrix usually constitutes between about 10 and about 90% by weight of the implant, about 40% to about 80% by weight of the implant, or at least about 10, at least about 20, at least about 30, at least about 40, at least about 50, at least about 60, at least about 70, at least about 80, or at least about 90 weight percent of the implant. In one variation, the biodegradable polymer matrix constitutes about 40% to 50% by weight of the implant. In one variation, the active agent (a non-steroidal anti-inflammatory agent such as ketorolac) is homogeneously dispersed in the biodegradable polymer of the implant.

Additional Agents

A drug delivery system, or extruded implant, or both may include one or more excipients to improve the properties of the system or implant. Useful excipients include buffering agents, preservatives, and electrolytes. Useful preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. Useful electrolytes include sodium chloride, potassium chloride, and magnesium chloride. These agents may be present in amounts of from 0.001 to about 5% by weight of the system or implant (% w/w). Suitable buffering agents include alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between about 2 to about 9 and more preferably about 4 to about 8. As such the buffering agent may be about 0.001 to about 5% by weight of the total implant.

In one variation, the implant does not comprise a polyvinyl alcohol.

Some implants may include a low molecular weight water soluble substance, such as a compound or material having a molecular weight of less than about 5,000 Daltons, less than about 1,000 Daltons, about 25 Daltons to about 500 Daltons, about 25 Daltons to about 400 Daltons, about 25 Daltons to about 300 Daltons, or about 25 Daltons to about 200 Daltons. Examples include, but are not limited to, a saccharide, e.g. as a monosaccharide, including a tetrose, a tetrulose, a pentose, a pentulose, a hexose such as dextrose, a hexulose, a heptose, a heptulose, an octose, an octulose, etc.; a disaccharide such as trehalose, sucrose, etc.; a sugar alcohol such as mannitol, galactitol, sorbitol; glycerol; or a salt, such as NaCl, KCl, Na₂SO₄, K₂SO₄, CaSO₄, MgSO₄, NH₄Cl, or phosphate salts. If present, the amount of low molecular weight water-soluble substance, may vary. For example, an implant may contain about 0.1% to about 10% or about 1% to about 10% of a saccharide or disaccharide, or other low molecular weight water-soluble substance, by weight.

Some implants may include a polyethylene glycol or polyethylene oxide, such as a polyethylene glycol or a polyethylene oxide having a molecular weight of about 300 Daltons to about 40,000 Daltons, about 300 Daltons to about 40,000 Daltons, about 1,000 Daltons to about 10,000 Daltons, about 3000 Daltons to about 40,000 Daltons, about 3350 Daltons, about 20,000 Daltons, or about 40,000 Daltons. For example, an implant may comprise a polyethylene glycol having a molecular weight of about 3350 (PEG 3350) or about 20,000 (PEG 20 k), which may be added at about 1% to about 20%, about 5% to about 10%, or about 10% by weight of the implant. This may help to modulate release of ketorolac. An implant may comprise a polyethylene glycol and a disaccharide.

In one variation, the present invention provides for an extruded biodegradable implant effective for treating pain and inflammation in an eye of a patient in need thereof, the implant comprising i) a biodegradable polymer matrix, and ii) ketorolac free acid or ketorolac tromethamine as the pharmaceutically active agent, wherein the implant comprises no pharmaceutically active agent other than ketorolac. The pain and inflammation may be post-operative pain and inflammation.

Release Kinetics

Release of an active agent such as ketorolac or other non-steroidal anti-inflammatory agent from a biodegradable polymer matrix may be a function of several processes, including diffusion out of the polymer, degradation of the polymer and/or erosion or degradation of the polymer. Some factors which influence the release kinetics of active agent from the implant can include the size and shape of the implant, the size of the active agent particles, the solubility of the active agent, the ratio of active agent to polymer(s), the method of manufacture, the surface area exposed, and the erosion rate of the polymer(s). For example, polymers may be degraded by hydrolysis (among other mechanisms), and therefore, any change in the composition of the implant that enhances water uptake by the implant will likely increase the rate of hydrolysis, thereby increasing the rate of polymer degradation and erosion, and thus, increasing the rate of active agent release.

The release kinetics of the implants described herein can be dependent in part on the surface area of the implants. A larger surface area may expose more polymer and active agent to ocular fluid, and may cause faster erosion of the polymer matrix and dissolution of the active agent particles in the fluid. Therefore, the size and shape of the implant may also be used to control the rate of release, period of treatment, and active agent concentration at the site of implantation. At equal active agent loads, larger implants will deliver a proportionately larger dose, but depending on the surface to mass ratio, may possess a slower release rate.

The release kinetics of active agent from an implant may be empirically determined using a variety methods. A USP approved method for dissolution or release test can be used to measure the rate of release (USP 23; NF 18 (1995) pp. 1790-1798). For example, using the infinite sink method, a weighed sample of the drug delivery system (e.g., implant) is added to a measured volume of a solution containing 0.9% NaCl in water (or other appropriate release medium such as phosphate buffered saline), where the solution volume will be such that the drug concentration after release is less than 20%, and preferably less than 5%, of saturation. The mixture is maintained at 37° C. and stirred slowly to ensure drug release. The amount of drug released as a function of time may be quantified by various methods known in the art, such as spectrophotometrically, HPLC, mass spectroscopy, etc.

Applications

The ketorolac-containing drug delivery systems described herein may be used to treat an ocular condition that affects or involves one or more parts or regions of the eye. Examples of ketorolac-containing drug delivery systems within the scope of the present invention include the extruded ketorolac-containing biodegradable implants described herein.

Examples of ocular conditions which may be treated by the present drug delivery systems include, but are not limited to, ocular inflammation and pain (inflammation and pain in an eye), such as, for example, post-operative ocular inflammation and pain; uveitis, macular edema, macular degeneration, retinal detachment, ocular tumors, bacterial, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, and vascular occlusion.

For example, the present drug delivery systems can be used to reduce ocular pain or inflammation or both following ocular surgery. The ocular surgery may be cataract surgery or refractive eye surgery, including incisional refractive surgery, or corneal refractive surgery. The ocular pain treatable with the present drug delivery systems may be perceived by the patient as a burning or stinging sensation. Refractive eye surgery is any eye surgery used to improve the refractive state of the eye and decrease or eliminate dependency on glasses or contact lenses. Refractive eye surgery can include surgical remodeling of the cornea. Examples of refractive eye surgery for the present invention include radial keratotomy, photorefractive keratectomy (PRK), and laser assisted sub-epithelial keratomileusis (LASEK). The method may comprise placing the drug delivery system in the eye during the surgery. For example, an extruded biodegradable ketorolac-containing implant according to the present invention may be placed into an opening in an eye during or immediately after the surgery. This may reduce inflammation related complications and may ensure compliance over a therapy of anti-inflammatory eye drops. For example, an implant can be placed into the anterior chamber of an eye during cataract surgery on the eye to thereby prevent and/or reduce inflammation associated with the cataract surgery. The implant may reduce inflammation of the iris and ciliary body (ICB) of the eye caused by the surgery (surgical trauma). The implants described herein can be configured to deliver ketorolac into the ICB in a manner that will effectively reduce or inhibit inflammation of the ICB that can follow cataract surgery. The iris refers to the pigmented tissue lying behind the cornea that gives color to the eye and that controls the amount of light entering the eye. The ciliary body includes the circumferential tissue inside the eye composed of ciliary muscle and ciliary processes that produce aqueous humor.

One embodiment provides for a method of reducing pain and inflammation in an eye of a mammal after cataract surgery on the eye, comprising placing a drug delivery system in the eye during cataract surgery. The method may involve placing the drug delivery system in an area of the eye opened by an incision in an eye, wherein the incision is made in the eye as part of a cataract surgery. In particular forms of this method, the drug delivery system is placed in the anterior chamber or posterior chamber of the eye during cataract surgery on the eye. This method may reduce and/or prevent pain and inflammation in the eye resulting from the surgery and may thereby reduce the risk of developing post-surgical complications arising from cataract surgery, such as macular edema. More generally, the method may reduce inflammation related complication from cataract surgery and may ensure improved compliance as compared to a therapy of anti-inflammatory eye drops.

As described above, the drug delivery system used in any of the foregoing methods may comprise or consist of an extruded ketorolac-containing biodegradable implant or plurality of microspheres. The implant may be sized and configured for placement in the anterior or posterior chamber.

Some methods for treating an inflammatory anterior segment condition comprise intracameral or subconjunctival administration of an implant described herein.

Some methods for treating persistent macular edema include (a) inserting a biodegradable implant into the anterior chamber, posterior chamber, or vitreous of a patient with persistent macular edema, the biodegradable implant comprising (i) ketorolac mixed with (ii) a biodegradable PLA polymer, PLGA co-polymer, or a combination thereof; (b) releasing at least a portion of, or substantially all of the ketorolac from the biodegradable implant; and (c) obtaining an improvement in the persistent macular edema.

In some embodiments, the drug delivery systems described herein, comprising an extruded implant or plurality of microspheres, may be used to treat an inflammation-mediated ocular condition. An inflammation-mediated ocular condition includes any condition of the eye which can benefit or potentially benefit from treatment with an anti-inflammatory agent such as ketorolac, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis, and uveal effusion.

In one embodiment, the invention provides for a method of reducing pain and/or inflammation in an eye of a mammal in need thereof, comprising the step of placing a biodegradable drug delivery system (such as, for example, and extruded biodegradable implant) as described herein in an ocular region of an eye of the mammal, thereby reducing inflammation and/or pain in the eye of the mammal.

With regard to any of the preceding methods, the mammal (and therefore patient) can be a human or non-human mammal, and the drug delivery system may be placed in the anterior chamber, posterior chamber, or vitreous body of the eye of the mammal during an ocular surgery such as cataract surgery, refractive eye surgery, incisional refractive surgery, or corneal refractive surgery, to thereby reduce the inflammation and/or pain associated with or caused by the surgery. The method may have the added benefit of enhancing post-operative repair of ocular tissue and reducing the risk of the patient developing macular edema after cataract surgery.

Methods of Implantation

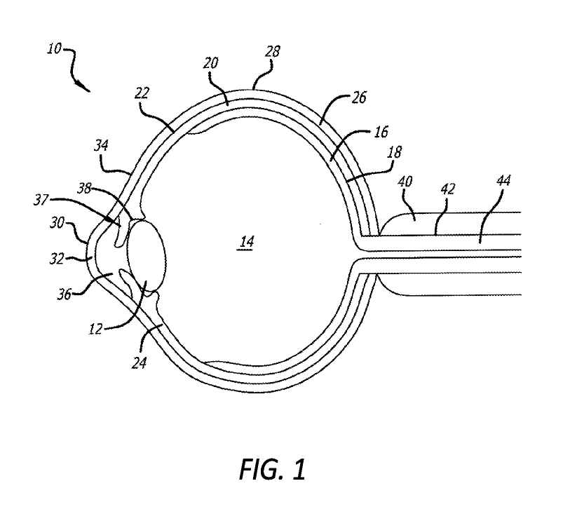

FIG. 1 illustrates a cross-sectional view of a human eye 10 in order to illustrate the various sites that may be suitable for implantation of an implant according to the present invention.

The eye 10 comprises a lens 12 and encompasses the vitreous chamber 14. Adjacent to the vitreous chamber is the optic part of the retina 16. Implantation may be into the vitreous 14, intraretinal 16 or subretinal 18. The retina 16 is surrounded by the choroid 20. Implantation may be intrachoroidal or suprachoroidal 22. Between the optic part of the retina and the lens, adjacent to the vitreous, is the pars plana 24. Surrounding the choroid 20 is the sclera 26. Implantation may be intrascleral 26 or episcleral 28. The external surface of the eye is the cornea 30. Implantation may be epicorneal 30 or intra-corneal 32. On the external surface of the eye is the conjunctiva 34. Behind the cornea is the anterior chamber 36, behind which is the lens 12. The posterior chamber 38 is just behind the iris 37 and surrounds the lens, as shown in the figure. Opposite from the external surface is the optic nerves, and the arteries and vein of the retina. Implants into the meningeal spaces 40, the optic nerve 42 and the intraoptic nerve 44 allows for drug delivery into the central nervous system, and provide a mechanism whereby the blood-brain barrier may be crossed. An intraocular implant may be placed in the anterior chamber 36 (also referred to as the intracameral space) or it may be administered behind the iris 37 into the posterior chamber 38. An implant may be inserted into the vitreous 14, the subconjunctival space 34, or subTenon's space 28.

An intraarticular implant may be implanted or injected into a joint such as a knee, an ankle, a shoulder, an elbow, a wrist, a hip, a spine, etc, to reduce pain and inflammation in the joint, occurring as a result of a medical condition such as arthritis or as a result of a surgery on the joint (i.e., post-operative pain and/or inflammation), such as arthroscopic surgery. To reduce post-operative pain and inflammation, the implant may be administered to the joint in a mammal before, during, or after a surgery on the joint.

The drug delivery systems, such as biodegradable implants or microspheres, can be inserted or placed into an ocular region of the eye, such as the anterior chamber, posterior chamber, or vitreous, by a variety of methods and devices, including placement by forceps, needle-equipped delivery devices, syringe, by a trocar, or by other types of applicators. The implant may be placed into the eye through an opening created by an incision (as during an ocular surgery) or may be directly injected or inserted into an ocular region using, for example, a needle-equipped delivery device containing the intraocular implant, or a trocar. One example of a device that may be used to insert the implants into an eye is disclosed in U.S. Patent Publication No. 2004/0054374. The method of placement may influence the therapeutic component or drug release kinetics. For example, delivering an implant with a trocar may result in placement of the implant deeper within the vitreous than placement by forceps, which may result in the implant being closer to the edge of the vitreous. The location of the implant may influence the concentration gradients of therapeutic component or drug surrounding the element, and thus influence the release rates (e.g., an element placed closer to the edge of the vitreous may result in a slower release rate). Microspheres of the present invention can be injected into the anterior chamber, posterior chamber, or vitreous of an eye using a needle or similar device.

In some embodiments, a method of treating a patient comprises administering one or more implants containing ketorolac to a patient by at least one of intravitreal injection, intracameral injection, posterior chamber injection, subconjunctival injection, sub-tenon injection, retrobulbar injection, and suprachoroidal injection. A syringe apparatus including an appropriately sized needle, for example, a 22, 25, 27, or 30 gauge needle, can be effectively used to inject the drug delivery system into the eye, such as the anterior or posterior chamber, of a human or non-human animal. Repeat injections are often not necessary due to the extended release of ketorolac from the systems.

In some embodiments, a hand held applicator is used to insert one or more biodegradable implants into the eye. The hand held applicator typically comprises an 18-30 gauge stainless steel needle, a lever, an actuator, and a plunger. Suitable devices for inserting an implant or implants into a posterior ocular region or site include those disclosed in United States Patent Application Publication No. US 2005/0101967.

For intracameral placement, an implant may be inserted at the time of cataract surgery or administered to the intracameral space via an applicator or injector system after or before a surgery or at any time as necessary to treat the pain and/or inflammation resulting from the surgery or to treat any other ocular condition.

The method of implantation generally first involves accessing the target area within the ocular region with the needle, trocar or implantation device. Once within the target area, e.g., the anterior chamber, posterior chamber, or vitreous cavity, a lever on a hand held device can be depressed to cause an actuator to drive a plunger forward. As the plunger moves forward, it can push the implant or implant into the target area.

Methods for Making Implants

A drug delivery system as described herein may comprise or consist of an extruded implant, compressed tablet, or plurality of microspheres. The implant, tablet, and microspheres can comprise ketorolac free acid or a pharmaceutically acceptable salt thereof and a biodegradable polymer matrix. The biodegradable polymer matrix can comprise one or more biodegradable polymers. Accordingly, the drug delivery system can be in different physical forms or geometric shapes including, but not limited to sclera plugs, extruded rods or filaments, sheets, films, or microspheres.

Various techniques may be employed to make implants. Useful techniques include phase separation methods, interfacial methods, extrusion methods (for example, hot melt extrusion), compression methods, pellet pressing, solvent casting, molding methods, injection molding methods, heat press methods and the like. Microspheres can be made by methods such as solvent evaporation, emulsion, spray drying, or precipitation. An extruded implant can be made by a sequential or double extrusion method. Choice of technique, and manipulation of technique parameters employed to produce the implants can influence the release rates of the drug. Room temperature compression methods may result in an implant with discrete microparticles of drug and polymer interspersed. Extrusion methods may result in implants with a progressively more homogenous dispersion of the drug within a continuous polymer matrix, as the production temperature is increased. The use of extrusion methods may allow for large-scale manufacture of implants and results in implants with a homogeneous dispersion of the drug within the polymer matrix. When using extrusion methods, the polymers and active agents that are chosen are stable at temperatures required for manufacturing. Extrusion methods may use temperatures of about 60° C. to about 150° C., or about 60° C. to about 130° C.

Different extrusion methods may yield implants with different characteristics, including but not limited to the homogeneity of the dispersion of the active agent within the polymer matrix. For example, using a piston extruder, a single screw extruder, and a twin screw extruder may produce implants with progressively more homogeneous dispersion of the active agent. When using one extrusion method, extrusion parameters such as temperature, feeding rate, circulation time, extrusion speed, die geometry, and die surface finish will have an effect on the release profile of the implants produced.

In one variation of producing implants by piston extrusion methods, the drug and polymer are first mixed at room temperature and then heated to a temperature range of about 60° C. to about 150° C., or about 130° C. for a time period of about 0 to about 1 hour, about 1 to about 30 minutes, about 5 minutes to about 15 minutes, or about 10 minutes. The implants are then extruded at a temperature of about 60° C. to about 130° C., or about 75° C.

In some screw extrusion methods, the powder blend of active agent and polymer is added to a single or twin screw extruder preset at a temperature of about 70° C. to about 130° C., and directly extruded as a filament or rod with minimal residence time in the extruder. The extruded filament or rod is then cut into small implants having the loading dose of active agent appropriate to treat the medical condition of its intended use.

In another aspect of the invention, kits for treating an ocular condition of the eye are provided, comprising: a) a container or package comprising an extended release implant or microspheres comprising ketorolac and a biodegradable polymer matrix; and b) instructions for use. Instructions may include steps of how to handle the drug delivery systems, how to insert the systems into an ocular region, and what to expect from using the systems.

In another embodiment, a drug delivery system, such as the implants disclosed herein, is administered to the anterior segment of an eye of a human or non-human animal patient. In one embodiment the implant is administered to the anterior chamber of the eye to reduce inflammation and/or pain associated with an ocular condition or surgery. In a particular embodiment, the implant is placed in the anterior chamber of the eye during ocular surgery, such as during cataract surgery to thereby reduce inflammation and/or pain relating to the surgery.

Ketorolac Implants

In some embodiments, the drug delivery system comprises about 10% to about 60%, about 20% to about 50%, about 20% to about 60%, about 20% to about 45%, about 20%, about 30%, about 35%, about 40%, about 45%, about 50%, or about 60% ketorolac by weight.

As described above, an intraocular drug delivery system can be in the form of a solid biodegradable implant (such as an extruded implant) comprising ketorolac or may comprise a plurality of biodegradable microspheres containing ketorolac.

Implants comprising ketorolac, either in salt or free acid form, may be useful for treatment of pain and/or inflammation, such as ocular or intraarticular inflammation occurring after a surgery, including an ocular surgery or intraarticular surgery. In addition, such an implant may also be useful for the treatment of macular edema, which may occur after cataract surgery, and for the treatment of chronic diabetic macular edema. In some embodiments, a biodegradable implant comprises about 10% to about 60% by weight ketorolac, or about 15% to about 60% by weight ketorolac, such as about 20%, 30%, about 40%, about 45%, or about 50% ketorolac tromethamine or ketorolac free acid by weight.

Some biodegradable implants may be capable of releasing a therapeutically effective amount of ketorolac, such as about 0.1 μg to about 100 μg of ketorolac tromethamine or ketorolac free acid per day, for about 2 weeks to about 6 months, about 2 weeks to about 3 months, for about 3 weeks, or about 2 weeks to about 6 weeks. In some embodiments, about 1 μg to about 5 μg, about 5 μg to about 400 μg of ketorolac, about 5 μg to about 200 μg, or about 5 μg to about 100 μg of ketorolac may be released in the first day after the implant is placed in the eye of a patient (e.g., within 24 hours after the implant is placed in an ocular region of the eye of a patient). A patient can be a human or non-human mammal. In some embodiments, the implant delivers at least 0.1 μg/day of ketorolac, or about 0.1 μg/day to about 5 μg/day, after the first day (i.e., after day 1). In some embodiments, the rate of delivery of ketorolac after the first day after the implant is placed in an eye or intraarticular region of the body continues for about 3 weeks to about 6 months, about 2 weeks to about 3 months, or about 2 weeks to about 6 weeks. The duration of release of ketorolac for these implants may vary depending upon factors such as the size of the implant and the amount of the ketorolac in the implant. Some drug delivery systems may be capable or releasing ketorolac free acid or salt for about 2 hours to about 2 years, about 1 day to about 1 year, about 1 week to about 1 year, about 3 months to about 6 months; and/or about 1 week, about 2 weeks, about 3 weeks, about 6 weeks, about 2 months, about 3 months, about 12 weeks, about 6 months, about 1 year, and/or any range bounded by, or between, any of these values. In some embodiments related to reducing inflammation after cataract surgery on an eye in a patient, an implant may deliver ketorolac for about 1 day to about 4 weeks, for about 3 weeks, or for about 6 weeks after the implant has been placed in the eye of the patient.

As described above, the drug delivery system may be an implant formed by an extrusion process (i.e., and extruded implant) and may be sized and configured for placement in the anterior chamber or posterior chamber of the eye.

The biodegradable polymer matrix of the drug delivery system (e.g., extruded implant) may comprise one biodegradable polymer or a mixture of two or more biodegradable polymers. For example, the implant may comprise a mixture of a first biodegradable polymer and a different second biodegradable polymer. One or more of the biodegradable polymers may have terminal acid groups (acid end groups; uncapped). Additionally, or alternatively, one or more of the biodegradable polymers in the matrix may have terminal ester groups (ester end groups; ester capped).

Useful biodegradable polymers include poly(D,L-lactide) polymers (PLAs) and poly(D,L-lactide-co-glycolide) copolymers (PLGAs). Specific examples of polymers that can be used individually or in combination to form the biodegradable polymer matrix of a ketorolac-containing sustained release intraocular drug delivery system according to the present disclosure include RESOMER® R203S, R203H, R202S, R202H, R207S, R208, RG502, RG502H, RG753S, and RG752S.

RESOMER® R203H is a poly(D,L-lactide) having an acid end group and an inherent viscosity of about 0.25-0.35 dl/g, as measured for a 0.1% solution in chloroform at 25° C.

RESOMER® R203S is a poly(D,L-lactide) having an ester end group and an inherent viscosity of about 0.25-0.35 dl/g, as measured for a 0.1% solution in chloroform at 25° C.

RESOMER® R202H is a poly(D,L-lactide) having an acid end group and an inherent viscosity of about 0.16-0.24 dl/g, as measured for a 0.1% solution in chloroform at 25° C.

RESOMER® R202S is a poly(D,L-lactide) having an ester end group and an inherent viscosity of about 0.16-0.24 dl/g, as measured for a 0.1% solution in chloroform at 25° C.

RESOMER® RG502 is a poly(D,L-lactide-co-glycolide) having an ester end group and an inherent viscosity of about 0.16-0.24 dl/g (as measured for a 0.1% solution in chloroform at 25° C.), and a D,L-lactide:glycolide ratio of about 50:50.

RESOMER® RG502H is a poly(D,L-lactide-co-glycolide) having an acid end group and an inherent viscosity of about 0.16-0.24 dl/g (as measured for a 0.1% solution in chloroform at 25° C.), and a D,L-lactide:glycolide ratio of about 50:50.

RESOMER® RG753S is a poly(D,L-lactide-co-glycolide) having an ester end group and an inherent viscosity of about 0.32-0.44 dl/g (as measured for a 0.1% solution in chloroform at 25° C.), and a D,L-lactide:glycolide ratio of about 75:25.

RESOMER® RG752S is a poly(D,L-lactide-co-glycolide) having an ester end group and an inherent viscosity of about 0.16-0.24 dl/g (as measured for a 0.1% solution in chloroform at 25° C.), and a D,L-lactide:glycolide ratio of about 75:25.

RESOMER® polymers are available from Evonik Industries AG, Germany. Ketorolac free acid and ketorolac tromethamine are available commercially from sources such as RECORDATI (Industria Chimica E Farmaceutica S.p.A, Via M, Civitali, 1-20148 Milano, Italia). The preparation of ketorolac is described in U.S. Pat. No. 6,197,976. PEG3350 is poly(ethylene glycol) with an average molecular weight of 3350 dalton. PEG 20K is poly(ethylene glycol) or poly(ethylene oxide) with an average molecular weight of 20,000 daltons.

In some embodiments, an extruded implant is placed in the anterior chamber, posterior chamber, or vitreous body of an eye in a mammal following or during an ocular surgery on the eye to thereby reduce or relieve inflammation or pain associated with the surgery or to treat the ocular condition. The ocular surgery may be cataract surgery or refractive eye surgery. In some forms of the invention, two implants, or more than two implants are placed in the eye (e.g., the anterior chamber) of the patient (mammal in need) to treat an ocular condition or reduce pain and inflammation in the eye of a patient.

In some embodiments, an extruded implant is placed in an ocular region of the eye of a patient to treat an ocular condition. The ocular condition can be an inflammatory condition such as inflammation of the iris and/or ciliary body of the eye, inflammation of the anterior segment of the eye, inflammation of the posterior segment of the eye, or uveitis. Or the ocular condition can be macular degeneration (including non-exudative age related macular degeneration and exudative age related macular degeneration); choroidal neovascularization; acute macular neuroretinopathy; macular edema (including cystoid macular edema and diabetic macular edema); Behcet's disease, diabetic retinopathy (including proliferative diabetic retinopathy); retinal arterial occlusive disease; central retinal vein occlusion; uveitic retinal disease; retinal detachment; retinopathy; an epiretinal membrane disorder; branch retinal vein occlusion; anterior ischemic optic neuropathy; non-retinopathy diabetic retinal dysfunction, retinitis pigmentosa and glaucoma. The implant(s) can be inserted into the anterior chamber, posterior chamber, or vitreous body. The implant may release a therapeutic amount of ketorolac to provide and retain a therapeutic effect for an extended period of time to thereby treat an ocular condition.

The present invention includes, but is not limited to, the following embodiments (1-28):

1. A biodegradable implant comprising a biodegradable polymer matrix and ketorolac free acid, ketorolac tromethamine, or a ketorolac prodrug associated with the biodegradable polymer matrix, wherein the implant releases at least 1% of its initial ketorolac load but no more than 50% of its initial ketorolac load during the first 60 minutes following placement of the implant in an eye of a mammal.

2. The implant of embodiment 1, wherein the implant is made by an extrusion process.

3. An implant according to embodiment 2, wherein the implant is configured for placement in the anterior chamber or posterior chamber of an eye.

4. An implant according to any of embodiments 1-3, wherein the implant comprises no pharmaceutically active agent other than ketorolac.

5. An implant according to any of embodiments 1-4, wherein the implant has a total weight of about 50 μg to about 500 μg and wherein the implant comprises about 20% to about 60% ketorolac by weight.

6. An implant according to any of embodiments 1-5, wherein the biodegradable polymer matrix comprises a poly(D,L-lactide-co-glycolide) copolymer and/or a poly(D,L-lactide) polymer.

7. An implant according to any of embodiments 1-6, wherein the implant releases about 5 μg to about 200 μg of ketorolac within 24 hours following placement in an eye and about 0 μg to about 5 μg of ketorolac/day thereafter (beginning on day 2) for about 1 day to about 6 weeks, for about two weeks or more, or for about 3 weeks or more after placement of the implant in the eye of a mammal.

8. An implant according to any of embodiments 1-7, wherein the biodegradable polymer matrix releases about 5 μg to about 200 μg of ketorolac within 24 hours following placement in an eye and about 0.001 μg to about 5 μg of ketorolac/day thereafter for about 1 day to about 6 weeks, for about two weeks or more, or for about 3 weeks or more after placement of the implant in the eye of a mammal.

9. An implant according to any of embodiments 1-8, wherein the biodegradable polymer matrix releases about 5 μg to about 200 μg of ketorolac within 24 hours following placement in an eye and about 0.01 μg to about 5 μg of ketorolac/day thereafter for about 1 day to about 6 weeks, for about two weeks or more, or for about 3 weeks or more after placement of the implant in the eye of a mammal.

10. An implant according to any of embodiments 1-9, wherein the biodegradable polymer matrix releases about 5 μg to about 200 μg of ketorolac within 24 hours following placement in an eye and about 0.05 μg to about 5 μg of ketorolac/day thereafter for about 1 day to about 6, for about two weeks or more, or for about 3 weeks or more weeks after placement of the implant in the eye of a mammal.

11. An implant according to any of embodiments 1-10, wherein the amount of ketorolac released within 24 hours (day 1) after placement of the implant in the eye is at least about 5 fold greater than the daily amount of ketorolac released after day 1.

12. An implant according to any of embodiments 1-11 further comprising about 0.1% to about 10% by weight of a polyethylene glycol or polyethylene oxide, said polyethylene glycol or polyethylene oxide having an average molecular weight of between about 300-40,000 daltons.

13. An implant according to any of embodiments 1-12 further comprising about 0.1% to about 10% trehalose, sucrose, mannitol, or dextrose.

14. An implant according to any of embodiments 1-13 wherein the implant is effective for reducing pain and/or inflammation in an eye associated with an ocular surgery of the eye in a mammal for a period of about 1 day to about 6 weeks, for about two weeks or more, or for about 3 weeks or more after placement of the implant in the eye of the mammal.

15. An implant according to any of embodiments 1-14 wherein the implant is effective for reducing pain and/or inflammation in the eye associated with an ocular surgery of the eye for about 2 weeks after placement of the implant in the eye.

16. An implant according to any of embodiments 1-15 wherein the implant is effective for reducing pain and/or inflammation in the eye associated with an ocular surgery of the eye for about 3 to 6 weeks after placement of the system in the eye.

17. An implant according to any of embodiments 14-16, wherein the ocular surgery is cataract surgery or refractive eye surgery.

18. A method for reducing post-operative inflammation and/or pain in an eye of a mammal, comprising placing an implant according to any of embodiments 1-17 into the anterior chamber of the eye during cataract surgery on the eye.

19. The method of embodiment 18, wherein the implant releases about 5 μg to about 200 μg ketorolac into the eye within 24 hours after the implant is inserted into the eye, and about 0.001 μg to about 5 μg of ketorolac/day thereafter for about 2 to 6 weeks after the implant is placed in the eye.

20. The method of embodiment 19, wherein the implant releases at least about 0.1 μg ketorolac/day for about 2 weeks to about 6 weeks beginning on day 2 after the implant is placed in the eye.

21. A method for reducing or relieving pain and/or inflammation in an eye of a mammal following cataract surgery, comprising placing an implant according to any of embodiments 1-17 in an ocular region of the eye receiving cataract surgery, thereby reducing pain and/or inflammation in the eye resulting from the surgery.

22. The method of embodiment 21, wherein the implant is placed in the anterior chamber or posterior chamber of the eye during cataract surgery on the eye.

23. A method of embodiment 22, whereby the implant is effective for reducing pain and inflammation in the eye for at least about 2 weeks after cataract surgery.

24. The method of embodiment 23, wherein the implant comprises about 10 μg to about 500 μg of ketorolac as ketorolac tromethamine, ketorolac free acid, or a combination of ketorolac tromethamine and ketorolac free acid.

25. The method of any of embodiments 18-24, wherein the implant comprises about 30% by weight ketorolac tromethamine, about 50% by weight of a poly(D,L-lactide) having an ester end group and an inherent viscosity of about 0.25-0.35 dl/g, as measured for a 0.1% solution in chloroform at 25° C. (RESOMER® R203S), and about 20% by weight of a poly(D,L-lactide) having an acid end group and an inherent viscosity of about 0.16-0.24 dl/g, as measured for a 0.1% solution in chloroform at 25° C. (RESOMER® R202H).

26. The method of any of embodiments 18-24, wherein the implant comprises about 45% by weight ketorolac free acid, about 25% by weight of a poly(D,L-lactide-co-glycolide) having an ester end group and an inherent viscosity of about 0.16-0.24 dl/g (as measured for a 0.1% solution in chloroform at 25° C.), and a D,L-lactide:glycolide ratio of about 50:50 (RESOMER® RG502), about 20% of a poly(D,L-lactide-co-glycolide) having an acid end group and an inherent viscosity of about 0.16-0.24 dl/g (as measured for a 0.1% solution in chloroform at 25° C.), and a D,L-lactide:glycolide ratio of about 50:50 (RESOMER® RG502H), about 5% by weight PEG 20,000 (PEG 20K), and about 5% trehalose.

27. A method of making an extruded biodegradable implant, comprising the step of extruding a mixture of a ketorolac and one or more biodegradable polymers to form a biodegradable material composite that will release an amount of ketorolac sufficient to reduce pain or inflammation in the eye for at least about two weeks after the composite is placed in the anterior chamber of the eye.

28. A method according claim 27, wherein the one or more polymer(s) is/are selected from the group consisting of polylactide polymers, poly (lactide-co-glycolide) polymers, and combinations thereof.

### Example 1

### Manufacture of Compressed Tablet Implants

Example 1 is a prophetic example. Ketorolac and biodegradable polymer(s) are accurately weighed and placed in a stainless steel mixing vessel. The vessel is sealed, placed on a Turbula mixer and mixed at a prescribed intensity, e.g., 96 rpm, and time, e.g., 15 minutes. The resulting powder blend is loaded one unit dose at a time into a single-cavity tablet press. The press is activated at a pre-set pressure, e.g., 25 psi, and duration, e.g., 6 seconds, and the tablet is formed and ejected from the press at room temperature.

### Example 2

### Manufacture of Extruded Implants

Example 2 is a prophetic example. Ketorolac and biodegradable polymer(s) are accurately weighed and placed in a stainless steel mixing vessel. The vessel is sealed, placed on a Turbula mixer and mixed at a prescribed intensity, e.g., 96 rpm, and time, e.g., 10-15 minutes. The resulting powder blend is fed into an Extruder (e.g., a DACA Microcompounder, Goleta, Calif.) and subjected to a pre-set temperature, e.g., 115° C., and screw speed, e.g., 12 rpm. The filament is extruded into a guide mechanism and cut to a desired length.

### Example 3

### Manufacture of Extruded Implants Using a Twin-Screw Extruder

As shown by the following examples, the release rate and release profile of ketorolac from extruded biodegradable implants may be modulated by altering the formulation parameters, such as the drug load, type of polymers, ratio of the polymers, and molecular weight of the polymers.

Biodegradable implants of ketorolac were fabricated by hot-melt extrusion method using a Haake twin-screw extruder. The fabrication process included 3 steps. (1) Powder blending: the components of each formulation were weighed and added to a blending jar together with two stainless steel balls. The jar was sealed and loaded onto a Turbula Mixer. The formulations were blended two times for 15 minutes each using a Turbula Mixer, with a manual mixing using a spatula between the two Turbula blendings; (2) Filament extrusion: the mixed powder formulations were fed through a force feeder and extruded using a Haake Minilab twin-screw extruder. The barrel and nozzle temperature was in the range of 75° C. to 120° C. and the diameter of the filaments was about 200 μm to about 480 μm; and (3) Cutting the filaments to a desired length: the extruded filaments were cut into implants with desired lengths using a blade.

A series of implants manufactured by this process (Example 3) are described in Tables 1 and 2. The weight percentage (% w/w) of ketorolac in each of the implants in Tables 1 and 2, below, is based on the total weight of the drug substance (ketorolac free acid or ketorolac tromethamine) initially added to the blending jar.

The rate of ketorolac release from each implant was measured in vitro by placing each implant into a glass scintillation vial with 10 mM phosphate buffered saline (PBS), pH7.4 (release medium). The glass scintillation vials were then placed in an incubator at 37° C. with shaking at 120 rpm. At given time points, the release medium was collected and entirely replaced with fresh medium. The concentration of ketorolac in the release medium was analyzed using HPLC.

TABLE 1
Ketorolac-containing Implants made according to Example 3.

| | | Composition (% w/w) | | | | | |
|---|---|---|---|---|---|---|---|
| Implant | Average implant | | PLA | PLA | PLGA | PLGA | PEG |
| No. (#) | weight* (μg) | Ketorolac | (R203S) | (R202H) | (RG502S) | (RG753S) | 3350 |
| 6-1 | 223 | 40 | 35 | 15 | 0 | 10 | 0 |
| | | Free acid | | | | | |
| 6-2 | 190 | 30 | 40 | 15 | 0 | 15 | 0 |
| | | Free acid | | | | | |
| 6-3 | 205 | 30 | 30 | 15 | 0 | 15 | 10 |
| | | Free acid | | | | | |
| 6-4 | 190 | 20 | 40 | 20 | 10 | 10 | 0 |
| | | Free acid | | | | | |
| 6-5 | 222 | 20 | 35 | 20 | 10 | 10 | 5 |
| | | Free acid | | | | | |
| 6-6 | 191 | 30 | 50 | 20 | 0 | 0 | 0 |
| | | Tromethamine salt | | | | | |

*Average of three implants. The implants in Table 1 had diameters of 350-370 μm and lengths of 1.8-2.2 mm.

TABLE 2
Ketorolac-containing Implants made according to Example 3

| Implant No. (#) and | Composition (% w/w) | | | | | | | |
|---|---|---|---|---|---|---|---|---|
| Average implant | | PLA | PLA | PLGA | PLGA | PEG | PEG | |
| weight* (μg) | Ketorolac | R202S | R202H | RG502 | RG502H | 3350 | 20K | Trehalose |
| 6-7 | 30 | 0 | 0 | 30 | 30 | 5 | 0 | 5 |
| (168 μg) | Free acid | | | | | | | |
| 6-8 | 50 | 0 | 0 | 0 | 46 | 4 | 0 | 0 |
| (114 μg) | Tromethamine salt | | | | | | | |
| 6-9 | 40 | 25 | 30 | 0 | 0 | 0 | 5 | 0 |
| (127 μg) | Free acid | | | | | | | |

*Average of three implants. Implants #6-7, #6-8, #6-9 in Table 2 had diameters of 350-370 μm, 240-260 μm, and 290-310 μm, respectively. Implants #6-7, #6-8, #6-9 had lengths of 1.4-1.6 mm, 1.9-2.1 mm, and 1.7-1.9 mm, respectively.

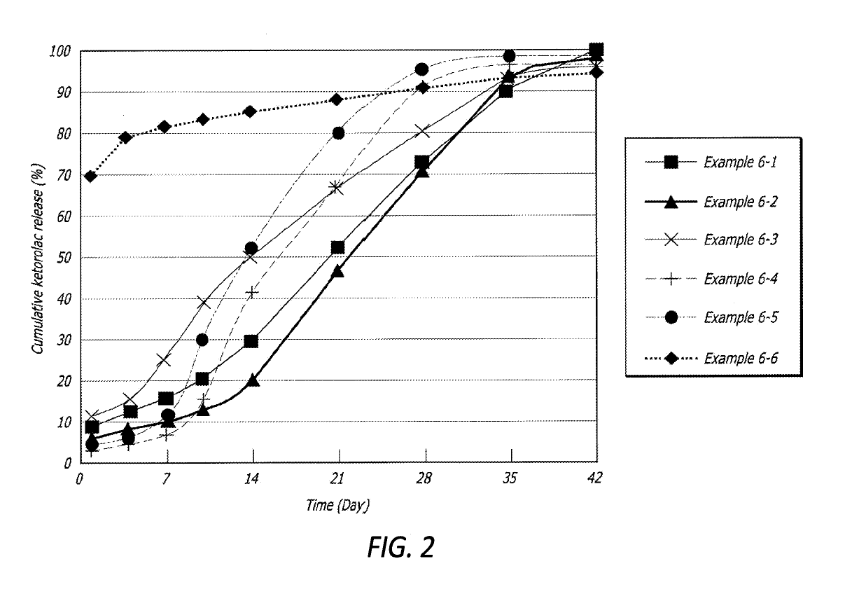

FIG. 2 shows the in vitro cumulative release profile of ketorolac for Implants 6-1 to 6-6.

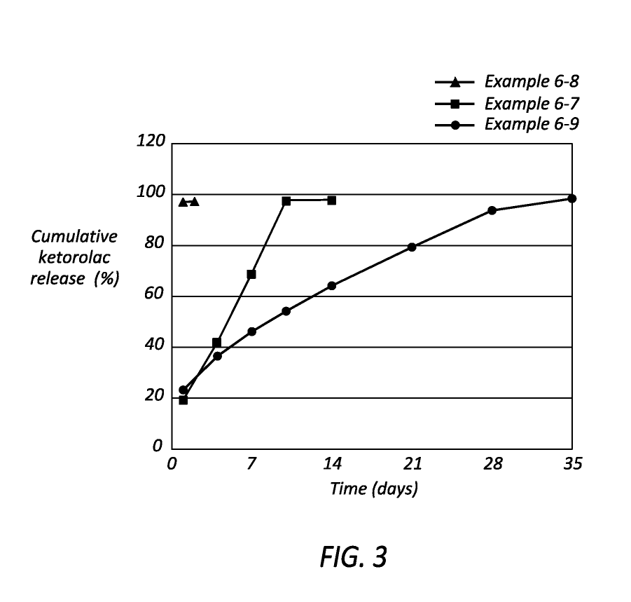

FIG. 3 shows the in vitro cumulative release profile of ketorolac for Implants 6-7 to 6-9.

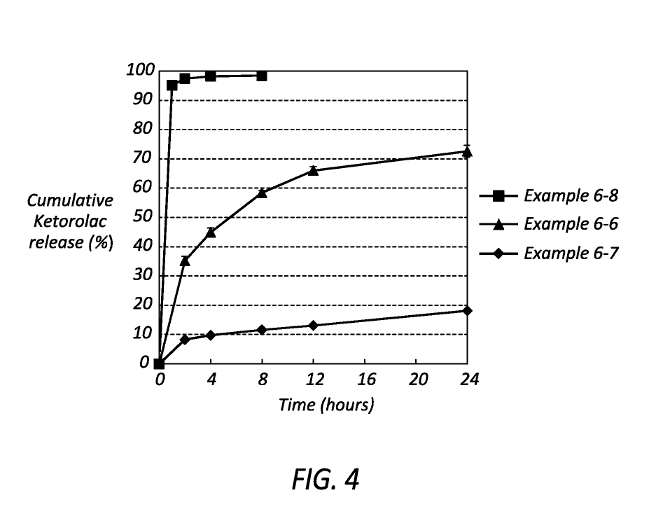

FIG. 4 shows the in vitro cumulative release profile of ketorolac during the first 24 hours in release medium for Implants 6-6 to 6-8.

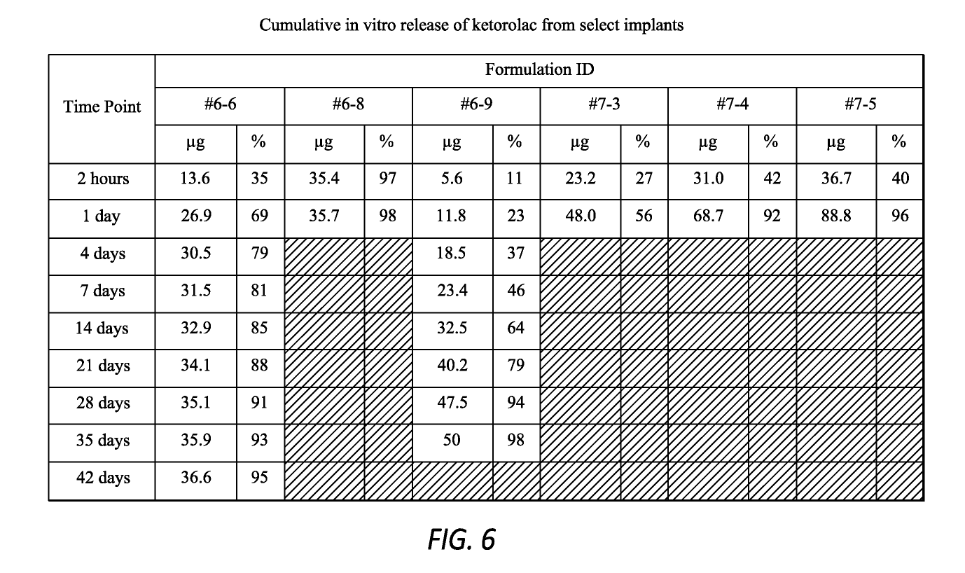

FIG. 6 shows the in vitro cumulative release data for Implants 6-6, 6-8, and 6-9.

### Example 4

### Manufacture of Extruded Implants Using a Piston Extruder

Biodegradable ketorolac-containing implants can also be fabricated using a piston extruder. The preparation process includes 4 steps. (1) Powder blending: weigh all the components of each formulation and add them into the blending jar together with 2 stainless steel balls. The jar is sealed and loaded onto a Turbula Mixer. The formulations are blended twice using the Turbula Mixer, each time for 15 minutes, with a manual mixing using a spatula in between the two turbula blendings; (2) Melt granulation: The mixed powder formulation is placed on a Teflon plate and heated at 90-120° C. in an oven for 5 minutes to melt. The melt is cooled down to room temperature and then ground using a mortar and pestle to make granules; (3) Filament extrusion: the granules are fed into a piston extruder through a stainless steel funnel and extruded at 80-120° C. The diameter of the filaments is about 360 μm; and (4) Cutting the filaments into implants with desired lengths using a blade.

A series of implants that were manufactured by this process (Example 4) are described in Tables 3 and 4. The weight percentage of ketorolac in the implants listed in Tables 3 and 4 is based on the total weight of the drug substance (ketorolac free acid or ketorolac tromethamine) initially added to the blending jar. The rate of ketorolac release from each of the implants was measured in vitro by placing each implant in a glass scintillation vial with 10 mM phosphate buffered saline (PBS), pH7.4 (release medium). The glass scintillation vials were then placed in an incubator at 37° C. with shaking at 120 rpm. At given time points, the release medium was collected and entirely replaced with fresh medium. The concentration of ketorolac in the release medium was analyzed using HPLC.

TABLE 3
Ketorolac-containing Implants made according to Example 4

| | Average | | | |
|---|---|---|---|---|
| | implant | Composition (% w/w) | | |
| Implant | weight* | | PLA | PLA |
| No. (#) | (μg) | Ketorolac | R203S | R203H |
| 7-1 | 372 | 30 | 70 | 0 |
| | | Free acid | | |
| 7-2 | 357 | 30 | 0 | 70 |
| | | Free acid | | |

*Average of three implants. The implants in Table 3 had diameters of 350-370 μm and lengths in the range of 2.9-3.2 mm.

TABLE 4
Ketorolac-containing Implants made according to Example 4

| | | Composition (% w/w) | | | | | | | |
|---|---|---|---|---|---|---|---|---|---|
| Implant | Average implant | | PLA | PLA | PLGA | PLGA | PLGA | PEG | |
| No. (#) | weight* (μg) | Ketorolac | R202S | R202H | RG502 | RG502H | RG753S | 20K | Trehalose |
| 7-3 | 190 | 45 | 0 | 0 | 25 | 20 | 0 | 5 | 5 |
| | | Free acid | | | | | | | |
| 7-4 | 186 | 40 | 20 | 0 | 0 | 15 | 20 | 5 | 0 |
| | | Free acid | | | | | | | |
| 7-5 | 186 | 50 | 0 | 0 | 0 | 45 | 0 | 5 | 0 |
| | | Free acid | | | | | | | |
| 7-6 | 190 | 45 | 0 | 0 | 25 | 30 | 0 | 0 | 0 |
| | | Free acid | | | | | | | |
| 7-7 | 190 | 40 | 40 | 20 | 0 | 0 | 0 | 0 | 0 |
| | | Tromethamine salt | | | | | | | |

*Average of three implants. The implants in Table 4 had diameters of 360-380 μm and lengths of 1.6-1.8 mm.

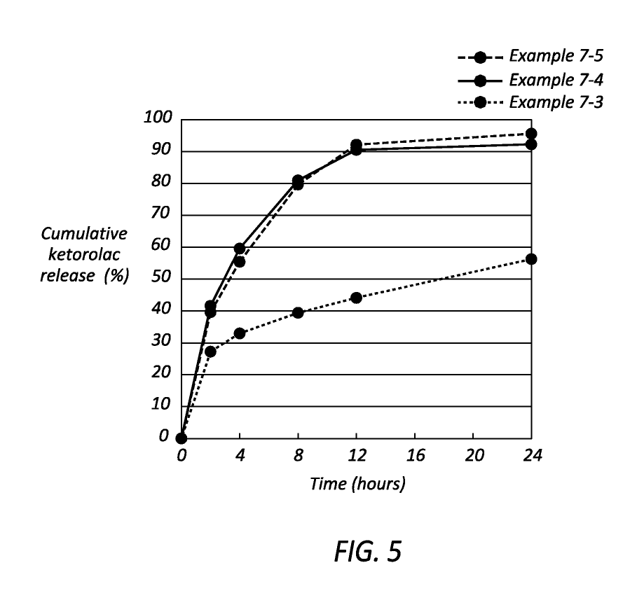

FIG. 5 shows the in vitro cumulative release profile of ketorolac for Implants 7-3 to 7-5 during the first 24 hours in release medium.

FIG. 6 shows the in vitro cumulative release data for implants 7-3, 7-4, and 7-5.

Implants 6-6 and 7-3 have desirable release profiles because these formulations provide an initial fast release of ketorolac followed by a substantially slower release. This type of release can be beneficial for treating ocular pain and inflammation such as that occurring after cataract surgery. The initial fast release can ensure sufficiently high tissue concentration immediately after surgery and therefore provide fast pain relief. The subsequent slow release can provide a maintenance dose for sustaining the therapeutic effect.

1. A biodegradable implant comprising a biodegradable polymer matrix and a pharmaceutical agent; wherein the biodegradable polymer matrix comprises a poly(D,L-lactide-co-glycolide) copolymer, a poly(D,L-lactide) polymer, or a combination thereof; and the pharmaceutical agent consists of a ketorolac selected from ketorolac free acid, ketorolac tromethamine, and a ketorolac prodrug;
wherein the implant:
(a) is made by an extrusion process;
(b) is configured for placement in the anterior chamber, posterior chamber, or vitreous body of an eye of a mammal;
(c) has a total weight of about 50 μg to about 500 μg;
(d) comprises about 20% to about 60% ketorolac by weight, and
(f) releases at least 1% of its initial ketorolac load but no more than 50% of its initial ketorolac load within about 60 minutes after placing the implant in the eye of the mammal.

2. The implant according to claim 1, wherein the biodegradable polymer matrix releases about 5 μg to about 200 μg of ketorolac within 24 hours after placing the implant in the eye, and about 0.001 μg to about 5 μg of ketorolac/day thereafter for about 1 day to about 6 weeks after placing of the implant in the eye of the mammal.

3. The implant according to claim 2, wherein the biodegradable polymer matrix releases about 5 μg to about 200 μg of ketorolac within 24 hours after placing the implant in the eye, and about 0.01 μg to about 5 μg of ketorolac/day thereafter for about 1 day to about 6 weeks after placing the implant in the eye of the mammal.

4. The implant according to claim 2, further comprising about 0.1% to about 10% by weight of a polyethylene glycol or polyethylene oxide, said polyethylene glycol or polyethylene oxide having an average molecular weight of between about 300 and about 40,000 daltons.

5. The implant according to claim 2, further comprising about 0.1% to about 10% trehalose, sucrose, mannitol or dextrose.

6. The implant according to claim 2, wherein the implant is effective for reducing pain, inflammation, or both, in the eye associated with an ocular surgery of the eye for a period of about 1 day to about 6 weeks after placing the implant in the eye of the mammal.

7. A method for reducing post-operative inflammation or pain in an eye of a mammal, the method comprising placing an implant according to claim 2 into an anterior chamber of the eye during cataract surgery on the eye.

8. The method of claim 7, wherein the implant releases about 5 μg to about 200 μg ketorolac into the eye within 24 hours after the implant is inserted into the eye, and about 0.001 μg to about 5 μg of ketorolac/day thereafter for about 2 to about 6 weeks after placing the implant in the eye.

9. The method of claim 8, wherein the implant releases at least about 0.1 μg ketorolac/day for about 2 weeks to about 6 weeks beginning on day 2 after placing the implant in the eye.

10. A method for reducing or relieving pain, inflammation, or both, in an eye of a mammal following cataract surgery, the method comprising placing an implant according to claim 2 in an ocular region of the eye receiving the cataract surgery, thereby reducing the pain, inflammation, or both, in the eye resulting from the surgery.

11. The method of claim 10, further comprising placing the implant in an anterior chamber, posterior chamber, or vitreous body of the eye during the cataract surgery on the eye.

12. The method of claim 11, whereby the implant reduces the pain, inflammation, or both, in the eye for at least about 2 weeks after the cataract surgery.

13. The method of claim 12, wherein the implant comprises:
(a) about 30% by weight ketorolac tromethamine;
(b) about 50% by weight of a poly(D,L-lactide) polymer having an ester end group and an inherent viscosity of about 0.25 to about 0.35 dl/g, as measured for a 0.1% solution in chloroform at 25° C.; and
(c) about 20% by weight of a poly(D,L-lactide) polymer having an acid end group and an inherent viscosity of about 0.16 to about 0.24 dl/g, as measured for a 0.1% solution in chloroform at 25° C.

14. The method of claim 12, wherein the implant comprises
(a) about 45% by weight ketorolac free acid;
(b) about 25% by weight of a poly(D,L-lactide-co-glycolide) polymer having an ester end group and an inherent viscosity of about 0.16 to about 0.24 dl/g, as measured for a 0.1% solution in chloroform at 25° C., and a D,L-lactide:glycolide ratio of about 50:50;
(c) about 20% of a poly(D,L-lactide-co-glycolide) having an acid end group and an inherent viscosity of about 0.16 to about 0.24 dl/g, as measured for a 0.1% solution in chloroform at 25° C., and a D,L-lactide:glycolide ratio of about 50:50;
(d) about 5% by weight polyethylene glycol having a molecular weight of about 20,000 daltons; and
(e) about 5% trehalose.
